(19) United States
(12) Patent Application Publication
(10) Pub. No.: US 2023/0109246 A1
(43) Pub. Date: Apr. 6, 2023

(54) MICROCHANNEL DEVICE

(71) Applicant: Shimadzu Corporation, Kyoto (JP)

(72) Inventor: Ippei TAKEUCHI, Kyoto-shi (JP)

(21) Appl. No.: 17/942,296

(22) Filed: Sep. 12, 2022

(30) Foreign Application Priority Data
Oct. 5, 2021 (JP) 2021-163827

(51) Int. Cl. B01L 3/00

(52) U.S. Cl. CPC B01L 3/502746; B01L 2200/0605; B01L 2300/0816; B01L 2300/0864; B01L 2400/088

(57) ABSTRACT

A microchannel device that can suppress a flow of a test solution produced between microchannels is provided. The microchannel device includes an opening for receiving a test solution that is to be injected therethrough, a main channel, a plurality of microchannels, a reservoir, an opening, and a gas permeable membrane. The plurality of microchannels include a first group and a second group. The microchannels included in each of the first group and the second group are arranged as being aligned in a direction of an X axis when the microchannel device is viewed in a plan view, and the first group and the second group are arranged as being aligned in a direction of a Y axis orthogonal to the direction of the X axis.

### BACKGROUND OF THE INVENTION

### Field of the Invention

The present disclosure relates to a microchannel device in a form of a plate used for a test in which a test solution containing a sample and an agent act on each other.

### Description of the Background Art

In order to test sensitivity or the like of bacteria to an antimicrobial, a test method using a microchannel device as in Japanese Patent Laying-Open No. 2017-67620 has been known. For example, in Japanese Patent Laying-Open No. 2017-67620, in a microchannel device including an introduction port and a discharge port that communicate with the outside and a channel through which a test solution supplied from the introduction port flows toward the discharge port, by injecting air into the channel from the introduction port, the test solution introduced previously is pressed into small channels. The channel is provided with a reaction portion where the test solution supplied from the introduction port is stored and an agent arranged in the reaction portion acts on bacteria.

### SUMMARY OF THE INVENTION

In filling a microchannel with a test solution, a method using a capillary action or a pressure application has been known. A method of injecting air as in Japanese Patent Laying-Open No. 2017-67620 is effective for reliably and quickly fill the microchannel with the test solution. When the test solution is injected from one introduction port into a microchannel device in which branching into a plurality of channels is made, however, there may be a difference in height of a fluid level (a fluid head) between channels due to a difference in distance from the introduction port to each channel. When there is a difference in height of the fluid level between the channels, the test solution injected in one channel may flow to another channel. When the test solution in one channel flows into the reaction portion provided in another channel, a correct result may not be observed.

The present disclosure was made to solve such a problem, and the present disclosure provides a microchannel device that can suppress a flow of a test solution produced between microchannels.

A microchannel device in the present disclosure is a microchannel device used for a test in which a test solution containing a sample and an agent are to act on each other, the microchannel device having a plate shape. The microchannel device includes a first opening for receiving the test solution that is to be injected therethrough, a main channel through which the injected test solution can flow, the main channel including an inlet-side end that communicates with the first opening and an outlet-side end located opposite to the inlet-side end, a plurality of microchannels each including a first-side end that communicates with the main channel and a second-side end located opposite to the first-side end, second openings that each communicate with the second-side end of each of the microchannels, a reservoir where the agent is to be stored, the reservoir being provided in each of the microchannels, and a gas permeable membrane that covers at least one of the second openings. The plurality of microchannels include a first group and a second group. The microchannels included in each of the first group and the second group are arranged as being aligned in a first direction when the microchannel device is viewed in a plan view. The first group and the second group are arranged as being aligned in a second direction orthogonal to the first direction.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram showing an exemplary construction of a microchannel device according to an embodiment.

FIG. 2 is a diagram showing an exemplary overall construction of a test apparatus according to the embodiment.

FIG. 3 is a diagram showing an exemplary construction around a pipet nozzle in the test apparatus according to the embodiment.

FIG. 4 is a block diagram for illustrating control of the test apparatus according to the embodiment.

FIG. 5 is a diagram showing an exemplary construction in which a test solution is injected into a channel in the microchannel device according to the embodiment.

FIG. 6 is a diagram showing a state after the test solution is injected into the channel in the microchannel device according to the embodiment.

FIG. 7 is a diagram showing a state after the test solution is discharged from a main channel in the microchannel device according to the embodiment.

FIG. 8 is a diagram for illustrating a method of discharging the test solution from the main channel in the microchannel device according to the embodiment.

FIG. 9 is a diagram showing a modification of the construction of the microchannel device.

FIG. 10 is a diagram showing another modification of the construction of the microchannel device.

### DESCRIPTION OF THE PREFERRED EMBODIMENTS

An embodiment will be described below in detail with reference to the drawings. The same or corresponding elements in the drawings have the same reference characters allotted and description thereof will not be repeated.

[Construction of Microchannel Device]

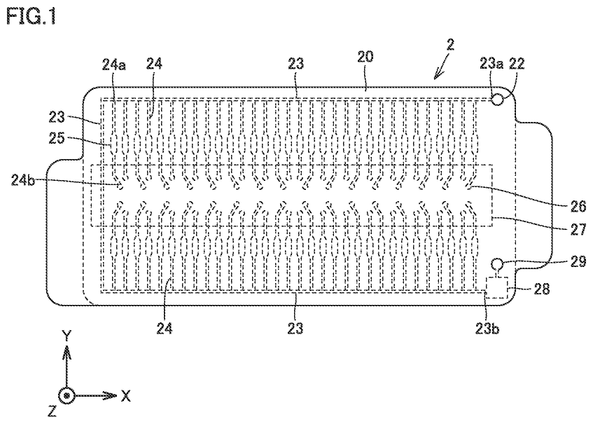

FIG. 1 is a diagram showing an exemplary construction of a microchannel device according to the embodiment. FIG. 1 shows a plan view of a microchannel device 2. Microchannel device 2 is placed on a table of a test apparatus which will be described later, and a test solution containing a sample is injected into each of a plurality of microchannels.

As shown in FIG. 1, microchannel device 2 includes a plate-shaped member 20 and a channel structure. The channel structure includes an opening 22 (a first opening), a main channel 23, a microchannel 24, a reservoir 25, an opening 26 (a second opening), a gas permeable membrane 27, a collection portion 28, and an opening 29 (a third opening). Microchannel device 2 does not have to include opening 26 (second opening).

Opening 22 is connected to one end of main channel 23 and communicates with main channel 23. The test solution is injected into main channel 23 from opening 22 by using a fluid pressure. The test solution injected into main channel 23 is further injected into microchannel 24. In the present embodiment, an air pressure is used as a fluid pressure. Opening 22 has a cross-section, for example, in an annular shape. Opening 22 has a diameter, for example, from 5 μm to 5 mm. In the present embodiment, one main channel 23 is connected to opening 22. One main channel 23 is arranged at a position surrounding an outer side of a plurality of microchannels 24.

Main channel 23 includes an inlet-side end 23a that communicates with opening 22 and an outlet-side end 23b located opposite to inlet-side end 23a. Main channel 23 that extends from opening 22 is further branched into a plurality of microchannels 24. Main channel 23 is connected to the plurality of microchannels 24 such that the test solution can flow thereto. The test solution that flows in from opening 22 flows to the plurality of branched microchannels 24 through main channel 23. Main channel 23 and microchannel 24 each have a rectangular cross-section, and main channel 23 and microchannel 24 each have a width, for example, from 1 μm to 1 mm. Main channel 23 and microchannel 24, however, are different from each other in depth (height). For example, main channel 23 has a depth of 0.5 mm, whereas microchannel 24 has a smaller depth of 0.025 mm. Therefore, microchannel 24 is higher in channel resistance than main channel 23. With microchannel 24 being higher in channel resistance than main channel 23, after main channel 23 is once filled with the test solution that flows in from opening 22 as will be described later, the test solution can flow into the plurality of microchannels 24 substantially at the same time.

When microchannel device 2 is viewed in a plan view as shown in FIG. 1, a lateral direction of the figure is defined as a direction of an X axis, a longitudinal direction of the figure is defined as a direction of a Y axis, and a direction of depth of the figure is defined as a direction of a Z axis. In the present embodiment, thirty-two microchannels 24 arranged as being aligned in the direction of the X axis are defined as one group, and two groups are arranged as being aligned in the direction of the Y axis. In other words, microchannel device 2 includes an upper group (a first group) and a lower group (a second group). A direction to which an arrow points along the Y axis is defined as an upper side. The plurality of microchannels 24 each include a first-side end 24a that communicates with main channel 23 and a second-side end 24b located opposite to first-side end 24a.

The plurality of microchannels 24 included in the upper group are each connected to main channel 23 arranged on an upper side of microchannel device 2. Therefore, the plurality of microchannels 24 included in the upper group are arranged in a direction downward along the Y axis, and the test solution branched from main channel 23 flows in the direction downward along the Y axis. The plurality of microchannels 24 included in the lower group are each connected to main channel 23 arranged on a lower side of microchannel device 2. Therefore, the plurality of microchannels 24 included in the lower group are arranged in a direction upward along the Y axis, and the test solution branched from main channel 23 flows in the direction upward along the Y axis.

After the plurality of microchannels 24 are branched from main channel 23, reservoir 25 is provided at a midpoint in each microchannel 24. Therefore, the test solution that flows in from opening 22 flows through main channel 23 and microchannel 24 to reservoir 25.

An agent is arranged in reservoir 25, and reservoir 25 is connected to opening 22 through main channel 23 and microchannel 24. The test solution that flows in from opening 22 is stored in reservoir 25. In reservoir 25, the test solution reacts with the agent. The agent is, for example, an antimicrobial. The agent may be a solid or a liquid. The agent is placed in advance in reservoir 25. In other words, before the test solution flows into reservoir 25, the agent is placed in reservoir 25. In the present embodiment, the agent is applied to entire reservoir 25.

Reservoir 25 is formed in a shape of a parallelepiped. Reservoir 25 has a side having a length, for example, from 10 μm to 10 mm.

In FIG. 1, sixty-four (=32×2) reservoirs 25 are formed in plate-shaped member 20. An identical volume of the test solution is stored in sixty-four reservoirs 25. A type and an amount of the agent provided in sixty-four reservoirs 25 may be identical or different.

Microchannel 24 is arranged further between reservoir 25 and opening 26. Microchannel 24 is arranged along the direction of the Y axis. Microchannel 24 has one end connected to reservoir 25 and has the other end (second-side end 24b) connected to opening 26. The test solution that flows in from reservoir 25 flows through microchannel 24 to opening 26.

Opening 26 is connected to the other end (second-side end 24b) of microchannel 24. Opening 26 has a cross-section, for example, in an annular shape. Opening 26 has a diameter, for example, from 5 μm to 5 mm.

Opening 26 is covered with gas permeable membrane 27. Specifically, in FIG. 1, thirty-two openings 26 connected to the plurality of microchannels 24 included in the upper group and thirty-two openings 26 connected to the plurality of microchannels 24 included in the lower group are arranged to face each other. Therefore, sixty-four (=32×2) openings 26 are arranged along the direction of the X axis in a central portion of microchannel device 2. Sixty-four openings 26 are covered with single gas permeable membrane 27. Instead of single gas permeable membrane 27 that covers sixty-four openings 26, two gas permeable membranes may separately cover thirty-two openings 26 included in the upper group and thirty-two openings 26 included in the lower group. Gas permeable membrane 27 may cover at least one of sixty-four openings 26.

Gas permeable membrane 27 performs a function to allow passage of gas and not to allow passage of liquid therethrough. Examples of a material for gas permeable membrane 27 include polytetrafluoroethylene (PTFE). Gas permeable membrane 27 is preferably water-repellent. Gas permeable membrane 27 has a thickness not larger than 1 mm.

Gas permeable membrane 27 is fixed to plate-shaped member 20 by adhesion by an adhesive or ultrasonic welding. Examples of the adhesive include a photocurable resin, a thermosetting resin, and a pressure-sensitive resin.

One main channel 23 connected to opening 22 is arranged to surround the outer side of microchannels 24 and connected to collection portion 28. Collection portion 28 is provided at outlet-side end 23b of main channel 23. Collection portion 28 is a portion where some of the test solution that flows into main channel 23 through opening 22 is collected. Collection portion 28 is formed in a shape of a parallelepiped. Collection portion 28 has a side having a length, for example, from 10 μm to 10 mm. Collection portion 28 may be provided with a member (water absorbing member) that absorbs moisture such as a sponge. A backflow from collection portion 28 to main channel 23 can thus be prevented and volatilization of the test solution from main channel 23 can be prevented.

Opening 29 is connected to an end of collection portion 28 opposite to an end connected to main channel 23. From opening 22 to opening 29, the test solution can flow through main channel 23 and collection portion 28. Opening 29 is closed by an opening and closing unit of the test apparatus which will be described later. By closing opening 29, the test solution that flows in main channel 23 is not discharged to collection portion 28 and opening 29, and by opening opening 29, the test solution that remains in main channel 23 can be discharged to collection portion 28 and collected therein.

[Apparatus Construction]

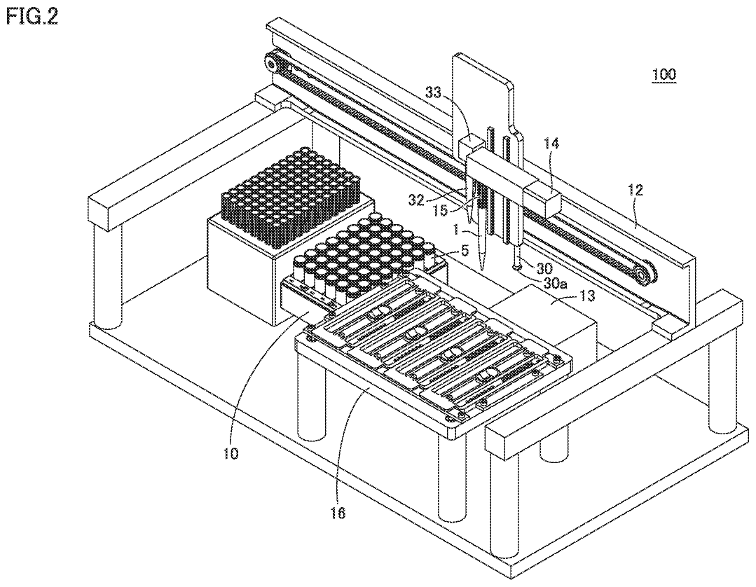

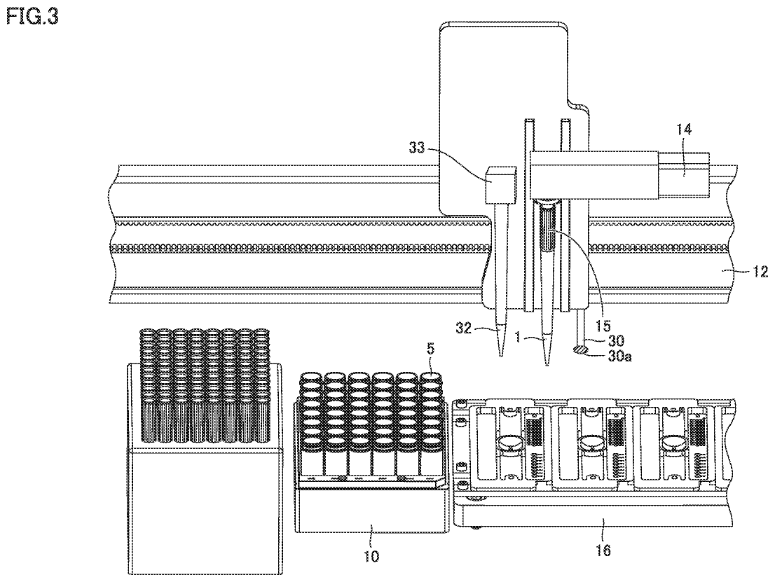

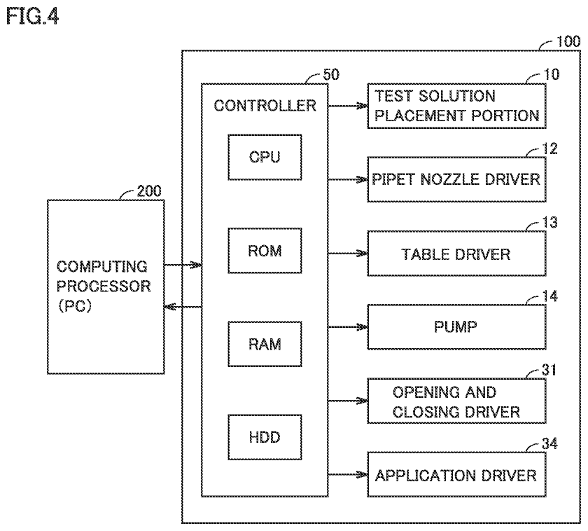

A test apparatus for conducting a test in which a test solution containing a sample and an agent act on each other with the use of microchannel device 2 will now be described. FIG. 2 is a diagram showing an exemplary overall construction of a test apparatus according to the embodiment. FIG. 3 is a diagram showing an exemplary construction around a pipet nozzle in the test apparatus according to the embodiment. FIG. 4 is a block diagram for illustrating control of the test apparatus according to the embodiment. The test apparatus in the present disclosure is an apparatus for measurement of a test solution containing a sample by injecting the test solution into a microchannel in microchannel device 2, and an example in which the test solution is injected into a microchannel for measuring sensitivity of bacteria to an antimicrobial (agent) is described below by way of example. The test solution contains a sample. The sample may be bacteria (pathogenic bacteria in a specific example). In the specific example, the test solution may be a suspension of bacteria. Naturally, for the test apparatus in the present disclosure, so long as a test solution is injected into a microchannel in microchannel device 2, limitation to the test solution described above is not intended.

Referring to FIGS. 2 to 4, a test apparatus 100 includes a test solution placement portion 10, a pipet nozzle driver 12, a table driver 13, a pump 14, a pipet nozzle 15, a table 16, an opening and closing unit 30, an opening and closing driver 31, an applicator 32, a pump 33, an application driver 34, and a controller 50.

Test solution placement portion 10 is a rack where a plurality of test solution containers 5 each containing a test solution can be arranged. In test solution placement portion 10, a plurality of test solution containers 5 can be set on test apparatus 100, in a unit of a rack.

Pipet nozzle 15 having a removable pipet chip 1 attached thereto suctions or discharges a test solution from test solution container 5 through a tip end of pipet chip 1. Pipet nozzle driver 12 horizontally and vertically moves pipet nozzle 15 and pump 14 connected to pipet nozzle 15. Pipet nozzle driver 12 can freely move pipet nozzle 15, for example, by means of a solenoid actuator or a stepping motor.

Table 16 is a support member on which microchannel device 2 is carried. Table 16 is in a shape of a flat plate and microchannel device 2 is fixed to an upper surface thereof. Table driver 13 can horizontally move table 16. Table driver 13 can freely move table 16, for example, by means of a solenoid actuator or a stepping motor. Naturally, table driver 13 may vertically move table 16 so that pipet nozzle 15 is not vertically moved. At least pipet nozzle driver 12 and table driver 13 are each a movement mechanism for changing a position of pipet nozzle 15 and a position of microchannel device 2 relative to each other.

Though not shown, pump 14 includes, for example, a syringe, a plunger capable of carrying out reciprocating motion within the syringe, and a drive motor that drives the plunger. Pump 14 can regulate an air pressure in pipet chip 1 by causing the plunger to carry out reciprocating motion while the plunger is connected to pipet nozzle 15 through a pipe to suction the test solution into pipet chip 1 or discharge the test solution in pipet chip 1 to the outside. Pump 14 can deliver air to the outside of pipet chip 1 by moving the plunger further into the syringe with the test solution in pipet chip 1 having been discharged to the outside.

Opening and closing unit 30 is a mechanism that opens or closes opening 29 (third opening) in microchannel device 2. Specifically, opening and closing unit 30 is a mechanism for closing the opening with an elastic member, and it is provided, for example, with a silicone resin 30a at a tip end of a rod-shaped support portion. Since opening and closing unit 30 is attached to pipet nozzle 15 at a predetermined position, it is moved to a position of opening 29 by moving pipet nozzle 15 to opening 22 (first opening) in microchannel device 2. Opening and closing driver 31 drives opening and closing unit 30 that has moved to the position of opening 29 to vertically move silicone resin 30a to press silicone resin 30a against opening 29 to close opening 29, and thus controls opening 29 to a closed state. Though test apparatus 100 shown in FIGS. 2 and 3 is provided with only a single opening and closing unit 30 in conformity with the construction of microchannel device 2, a plurality of opening and closing units 30 may be provided in accordance with the number of openings 29 to be opened and closed. Opening and closing driver 31 may not only vertically move silicone resin 30a but also move opening and closing unit 30 relative to pipet nozzle 15.

Applicator 32 applies a sealing material to opening 22 or 29 in microchannel device 2 in order to suppress volatilization of the injected test solution from that opening. Specifically, applicator 32 is implemented, for example, by a nozzle that discharges the sealing material such as silicone oil to the opening or the like, and applies the sealing material to the opening or the like from the nozzle by means of pump 33. The construction of applicator 32 is not limited as such, and a mechanism that applies the sealing material to the opening or the like with a brush may be applicable. Application driver 34 moves applicator 32 to a position of opening 22 or 29 in microchannel device 2 to which the sealing material is to be applied and drives pump 33. Though FIGS. 2 and 3 show the construction in which applicator 32 and pipet nozzle 15 are provided in the same movement mechanism, applicator 32 may be provided in a movement mechanism different from a movement mechanism where pipet nozzle 15 is provided and application driver 34 may move applicator 32. Unless volatilization of the test solution gives rise to a problem, test apparatus 100 does not have to include applicator 32.

Controller 50 controls an operation of test apparatus 100. Controller 50 includes a processor such as a central processing unit (CPU) and a memory such as a read only memory (ROM) and a random access memory (RAM). A control program is stored in the memory. The processor controls the operation of test apparatus 100 by execution of the control program. The memory of controller 50 may include a hard disk drive (HDD).

Controller 50 controls a motor of table driver 13 to move table 16 such that microchannel device 2 is located at a prescribed position. Controller 50 controls a motor of pipet nozzle driver 12 to move pipet nozzle 15 in order to inject the test solution into opening 22 of microchannel 24 in microchannel device 2 after microchannel device 2 is moved to the prescribed position. Furthermore, controller 50 controls opening and closing driver 31 to switch between opening and closing of opening 29 in microchannel device 2. Controller 50 controls application driver 34 to apply the sealing material to opening 22 or 29 or the like in microchannel device 2.

Specifically, controller 50 controls the motor of pipet nozzle driver 12 to move pipet nozzle 15 to a position of prescribed test solution container 5, and controls pump 14 to suction the test solution in test solution container 5 from the tip end of pipet chip 1. Thereafter, controller 50 controls the motor of pipet nozzle driver 12 to move pipet nozzle 15 to the position of opening 22 in microchannel device 2, and controls pump 14 to inject the test solution into opening 22 from the tip end of pipet chip 1.

Controller 50 can be connected to a computing processor 200 implemented by a personal computer (PC) or a dedicated computer. A user can manage test apparatus 100 by means of computing processor 200. For example, with computing processor 200, an amount of movement of table 16 by table driver 13, an amount of movement of pipet nozzle 15 by pipet nozzle driver 12, and an amount of the test solution suctioned or discharged from the tip end of pipet chip 1 by pump 14 can be set. Computing processor 200 may electrically be connected to another apparatus arranged adjacently to test apparatus 100 to configure a test system.

[Injection of Test Solution into Microchannel Device]

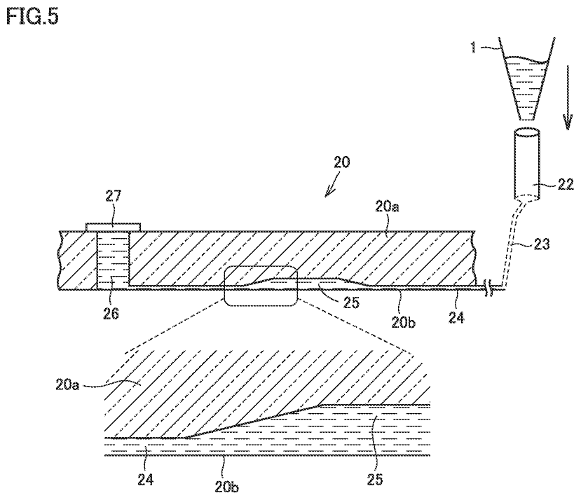

A method of injecting a test solution into microchannel device 2 with the use of test apparatus 100 will now be described. FIG. 5 is a diagram showing an exemplary construction in which the test solution is injected into the channel in microchannel device 2 according to the embodiment. Plate-shaped member 20 of microchannel device 2 includes a first plate-shaped member 20a on an upper side in FIG. 5 and a second plate-shaped member 20b on a lower side therein. Second plate-shaped member 20b is layered on first plate-shaped member 20a. Second plate-shaped member 20b is arranged in a negative direction (a downward direction) of the Z axis shown in FIG. 1 with respect to first plate-shaped member 20a.

First plate-shaped member 20a and second plate-shaped member 20b are each formed of a transparent material into a shape of a rectangular plate. Examples of the material for first plate-shaped member 20a and second plate-shaped member 20b include an acrylic resin such as polymethyl methacrylate and glass. A channel structure is formed in first plate-shaped member 20a. Specifically, in first plate-shaped member 20a, opening 22, main channel 23, microchannel 24, reservoir 25, opening 26, and collection portion 28 (see FIG. 1) are provided. Second plate-shaped member 20b functions as a lower surface for opening 22, main channel 23, microchannel 24, reservoir 25, opening 26, and collection portion 28. A thickness of first plate-shaped member 20a and second plate-shaped member 20b is set, for example, to 0.5 mm to 3 mm, although it is not particularly limited. Though second plate-shaped member 20b is directly fixed to first plate-shaped member 20a by ultrasonic welding, it may be fixed by an adhesive.

In the present embodiment, the test solution suctioned by pipet chip 1 is injected into microchannel 24 through opening 22 by application of an air pressure. As shown in FIG. 5, the test solution that flows in from pipet chip 1 flows through opening 22 and main channel 23, and microchannel 24, reservoir 25, and opening 26 are filled therewith. In microchannel device 2, however, the plurality of microchannels 24 communicate with one another through main channel 23. Therefore, when the test solution is injected from single opening 22 through main channel 23 into microchannel device 2 in which branch into the plurality of microchannels 24 is made, a height of a fluid level (fluid head) is different between channels and the difference causes a flow of the test solution between the channels.

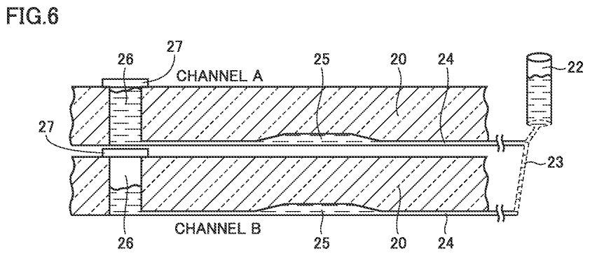

FIG. 6 is a diagram showing a state after the test solution is injected into the channel in microchannel device 2 according to the embodiment. An upper path shown in FIG. 6 is denoted as a path A and a lower path is denoted as a path B. The test solution that flows in from opening 22 passes through main channel 23 and is divided into the test solution in microchannel 24 along path A and the test solution in microchannel 24 along path B, and reaches openings 26. As shown in FIG. 6, path B is longer in distance from opening 22 than path A. Therefore, the fluid head at opening 26 of path A is higher than the fluid head at opening 26 of path B. Since there is a difference in fluid head between path A and path B, a flow of the test solution for eliminating the difference is produced between path A and path B. When a flow of the test solution is produced in reservoirs 25 in path A and path B, a correct result may not be observed.

In the present embodiment, after the test solution is injected into the plurality of microchannels 24, the test solution that remains in main channel 23 is discharged to collection portion 28. By discharging the test solution that remains in main channel 23, each channel becomes independent such that the plurality of microchannels 24 do not behave as a single channel through main channel 23 to prevent the flow caused by the difference in fluid head.

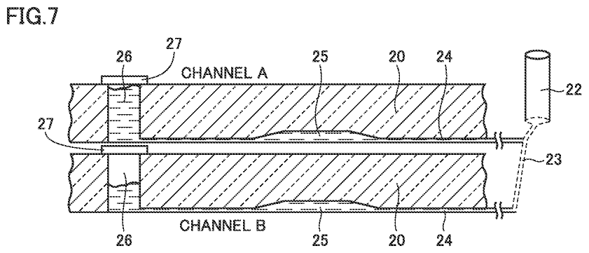

FIG. 7 is a diagram showing a state after the test solution is discharged from main channel 23 in microchannel device 2 according to the embodiment. An upper path shown in FIG. 7 is denoted as a path A and a lower path is denoted as a path B. By discharging the test solution from main channel 23, microchannel 24 along path A and microchannel 24 along path B do not behave as a single channel through main channel 23. Therefore, even when the fluid head at opening 26 of path A is higher than the fluid head at opening 26 of path B, a flow of the test solution for eliminating the difference is not produced between path A and path B.

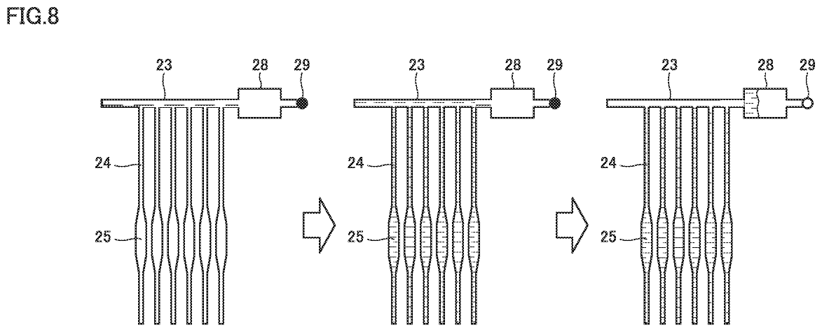

FIG. 8 is a diagram for illustrating a method of discharging the test solution from main channel 23 in microchannel device 2 according to the embodiment. In FIG. 8, the plurality of microchannels 24 are connected to main channel 23 and collection portion 28 is provided at one end (outlet-side end 23b) of main channel 23. Opening 29 is provided at an end of collection portion 28 opposite to the end connected to main channel 23. Though not shown, main channel 23 is connected to opening 22 at the end opposite to the end connected to collection portion 28.

Each microchannel 24 is higher in channel resistance than main channel 23. Therefore, when the test solution flows into main channel 23, unless main channel 23 is completely filled with the test solution, the test solution does not flow into each microchannel 24. In order for each microchannel 24 to be higher in channel resistance than main channel 23, main channel 23 should be larger in cross-sectional area than each microchannel 24. When main channel 23 is equal in width to each microchannel 24, main channel 23 is made larger in depth than each microchannel 24. For example, by setting the depth of each microchannel 24 to 0.001 mm with the depth of main channel 23 being set to 0.5 mm, the cross-sectional area of main channel 23 can be five hundred times as large as that of each microchannel 24.

When the test solution flows into main channel 23, opening 29 is closed by silicone resin 30a of opening and closing unit 30. Therefore, the test solution that flows into main channel 23 is not discharged to collection portion 28 at this stage. After main channel 23 is completely filled with the test solution, the test solution flows into microchannels 24 substantially at the same time as shown in FIG. 8. The test solution thus flows into each microchannel 24 and each reservoir 25.

Thereafter, silicone resin 30a of opening and closing unit 30 that closes opening 29 is removed and air is sent from opening 22 while opening 29 is open, so that the test solution that remains in main channel 23 is discharged to collection portion 28 as shown in FIG. 8. Air discharged from pipet chip 1 for injecting the test solution can be used as air sent from opening 22. Collection portion 28 includes a space where the test solution in main channel 23 to be discharged is held (buffer space), and the space is larger than a volume of main channel 23.

Whether or not to discharge the test solution in main channel 23 to collection portion 28 can be controlled by opening and closing of opening 29. The test solution that remains in main channel 23 is discharged from opening 22 to collection portion 28 by means of air.

In the present embodiment, the test solution is injected into each microchannel 24 and the test solution that remains in main channel 23 is discharged to collection portion 28. Thereafter, a sealing material such as silicone oil is applied to opening 22, opening 26, and opening 29. The sealing material to be applied to opening 22, opening 26, and opening 29 is not limited to silicone oil 33a, and any material is applicable so long as the material rests at opening 22, opening 26, and opening 29 and suppresses volatilization of the test solution.

As shown in FIG. 1, microchannel device 2 is constructed such that all microchannels 24 communicate with single main channel 23. Therefore, the test solution collected in collection portion 28 is only the test solution that remains within single main channel 23. Depending on the construction of the microchannel device, however, a plurality of main channels may be provided and a plurality of microchannels 24 may separately communicate with each main channel. For example, in an example where four main channels communicate with the opening (first opening) in which the test solution is injected, a slight difference in channel resistance is produced even though the main channels are identical in shape to one another. When air is injected through the opening (first opening) to the four main channels different in channel resistance from one another in order to discharge the test solution, there may remain one main channel from which the test solution is not successfully discharged, depending on a condition of injected air.

When the test solution remains in one of the four main channels, in spite of injection of air through the opening (first opening) thereafter, air escapes through the main channels from which the test solution was successfully discharged, and the test solution cannot be discharged from the main channel where the test solution remains.

In the plurality of microchannels 24 that communicate with the main channel where the test solution remains, the flow of the test solution is produced between channels for eliminating the difference in fluid head between the channels as described above.

In the present embodiment, the number of main channels 23 is made equal to the number of openings 22 (first openings) in which the test solution is injected, and microchannels 24 included in each of a plurality of groups are arranged to all communicate with single main channel 23. In other words, rather than providing a single linearly extending channel and making all microchannels communicate with that channel, main channel 23 is arranged to connect through one channel, a plurality of microchannels 24 arranged on plate-shaped member 20 as being divided into a plurality of groups. When all microchannels communicate with the linearly extending channel, one side of the microchannel device is approximately twice as long as one side of microchannel device 2 shown in FIG. 1, which necessitates increase in size of a test apparatus for testing or a refrigerator for storage. By arranging a single main channel 23 as in microchannel device 2, the test solution in main channel 23 can all be discharged to collection portion 28 without change in outer dimension.

In microchannel device 2 shown in FIG. 1, a plurality of openings 26 included in the upper group and a plurality of openings 26 included in the lower group are arranged to face each other. Therefore, since all openings 26 are arranged along the direction of the X axis in the central portion of microchannel device 2, single gas permeable membrane 27 can cover all openings 26 and the number of gas permeable membranes 27 can also be reduced.

[Modification of Microchannel Device]

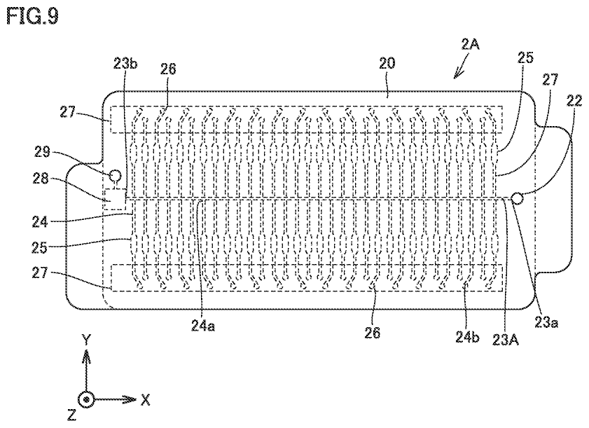

The construction of the microchannel device is not limited to the construction shown in FIG. 1. FIG. 9 is a diagram showing a modification of the construction of the microchannel device. A microchannel device 2A shown in FIG. 9 includes plate-shaped member 20 and a channel structure. The channel structure includes opening 22 (first opening), a main channel 23A, microchannel 24, reservoir 25, opening 26 (second opening), gas permeable membrane 27, collection portion 28, and opening 29 (third opening). Elements of microchannel device 2A identical or corresponding to those of microchannel device 2 shown in FIG. 1 have the same reference numerals allotted and description thereof will not be repeated.

In microchannel device 2A, single main channel 23A is arranged along the direction of the X axis in the central portion of microchannel device 2A. Main channel 23A has one end connected to opening 22 and has the other end connected to collection portion 28. A plurality of microchannels 24 each communicate with main channel 23A arranged in the central portion. Specifically, the plurality of microchannels 24 included in the upper group are arranged in the direction upward along the Y axis from a portion of communication with main channel 23A, and the test solution branched from main channel 23A flows in the direction upward along the Y axis. The plurality of microchannels 24 included in the lower group are arranged in the direction downward along the Y axis from a portion of communication with main channel 23A, and the test solution branched from main channel 23A flows in the direction downward along the Y axis. Main channel 23A in microchannel device 2A can be shorter than main channel 23 in microchannel device 2.

In FIG. 9, thirty-two openings 26 connected to the plurality of microchannels 24 included in the upper group and thirty-two openings 26 connected to the plurality of microchannels 24 included in the lower group are arranged on respective sides of opposing sides of microchannel device 2A. Therefore, two gas permeable membranes 27 in total, one covering thirty-two openings 26 included in the upper group and one covering thirty-two openings 26 included in the lower group, are required.

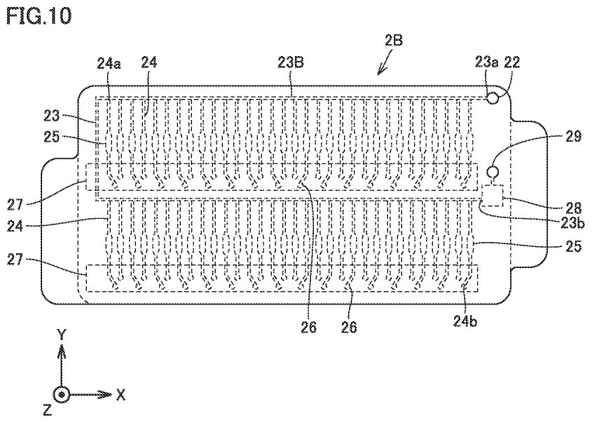

FIG. 10 is a diagram showing another modification of the construction of the microchannel device. A microchannel device 2B shown in FIG. 10 includes plate-shaped member 20 and a channel structure. The channel structure includes opening 22 (first opening), a main channel 23B, microchannel 24, reservoir 25, opening 26 (second opening), gas permeable membrane 27, collection portion 28, and opening 29 (third opening). Elements of microchannel device 2B identical or corresponding to those of microchannel device 2 shown in FIG. 1 have the same reference numerals allotted and description thereof will not be repeated.

In microchannel device 2B, single main channel 23B is arranged at a position that passes on the outer side of the upper group and passes through the central portion of microchannel device 2B. Main channel 23B has one end connected to opening 22 and has the other end connected to collection portion 28. Microchannels 24 in the upper group communicate with main channel 23B arranged on the outer side and microchannels 24 in the lower group communicate with main channel 23B arranged in the central portion. Specifically, the plurality of microchannels 24 included in the upper group are arranged in the direction downward along the Y axis from a portion of communication with main channel 23B on the outer side, and the test solution branched from main channel 23B flows in the direction downward along the Y axis. The plurality of microchannels 24 included in the lower group are arranged in the direction downward along the Y axis from a portion of communication with main channel 23B in the central portion, and the test solution branched from main channel 23B flows in the direction downward along the Y axis. Main channel 23B in microchannel device 2B can be shorter than main channel 23 in microchannel device 2.

In FIG. 10, thirty-two openings 26 connected to the plurality of microchannels 24 included in the upper group are arranged in the central portion of microchannel device 2B and thirty-two openings 26 connected to the plurality of microchannels 24 included in the lower group are arranged on a side of one side of microchannel device 2B. Therefore, two gas permeable membranes 27 in total, one covering thirty-two openings 26 included in the upper group and one covering thirty-two openings 26 included in the lower group, are required.

[Other Modifications]

(1) In test apparatus 100 according to the embodiment, opening 29 is closed by silicone resin 30a of opening and closing unit 30. Without being limited as such, any construction to switch between opening and closing of opening 29 may be applicable. For example, when an opening and closing mechanism (a shutter etc.) is provided in advance at opening 29 of microchannel device 2, opening and closing unit 30 may be constructed to switch a state of the opening and closing mechanism.

(2) In test apparatus 100 according to the embodiment, the sealing material is described as being applied to openings 22, 26, and 29. Without being limited as such, any construction may be applicable so long as volatilization of the test solution can be suppressed. For example, volatilization of the test solution may be suppressed by attaching a prepared cover to openings 22, 26, and 29.

(3) Opening 29 has an annular cross-section and communicates with collection portion 28. Therefore, when the test solution that remains in main channel 23 is discharged to collection portion 28 by sending air from opening 22 while opening 29 is open, depending on the pressure of sent air, the test solution is not only discharged to collection portion 28 but also may flow over opening 29. Then, opening 29 may be covered with a gas permeable membrane.

In FIG. 1, opening 29 is provided at the end of collection portion 28 opposite to the end to which main channel 23 is connected. By providing an opening in collection portion 28 itself, however, opening 29 does not have to be provided. Furthermore, the opening provided in collection portion 28 may be covered with a gas permeable membrane. Collection portion 28 itself does not have to be provided at outlet-side end 23b of main channel 23.

[Aspects]

The embodiment described above is understood by a person skilled in the art as specific examples of aspects below.

(Clause 1)

A microchannel device according to one aspect is a microchannel device used for a test in which a test solution containing a sample and an agent are to act on each other, the microchannel device having a plate shape, the microchannel device including: a first opening for receiving the test solution that is to be injected therethrough; a main channel through which the injected test solution can flow, the main channel including an inlet-side end that communicates with the first opening and an outlet-side end located opposite to the inlet-side end; a plurality of microchannels each including a first-side end that communicates with the main channel and a second-side end located opposite to the first-side end; second openings that each communicate with the second-side end of each of the microchannels; a reservoir where the agent is to be stored, the reservoir being provided in each of the microchannels; and a gas permeable membrane that covers at least one of the second openings, wherein the plurality of microchannels include a first group and a second group, the microchannels included in each of the first group and the second group are arranged as being aligned in a first direction when the microchannel device is viewed in a plan view, and the first group and the second group are arranged as being aligned in a second direction orthogonal to the first direction.

According to the microchannel device described in Clause 1, the test solution in the main channel can all be discharged, and hence the flow of the test solution produced between microchannels can be suppressed.

(Clause 2)

The microchannel device described in Clause 1, further including a collection portion where some of the test solution is collected, the collection portion being provided at the outlet-side end, and a third opening provided in the collection portion.

According to the microchannel device described in Clause 2, the test solution discharged from the main channel can all be collected in the collection portion.

(Clause 3)

The microchannel device described in Clause 1 or 2, wherein when the microchannel device is viewed in the plan view, the second openings included in the first group and the second openings included in the second group are arranged to face each other, and a single main channel is arranged at a position surrounding an outer side of the plurality of microchannels.

According to the microchannel device described in Clause 3, the second openings included in the first group and the second openings included in the second group are arranged to face each other, and hence the number of gas permeable membranes 27 can be reduced.

(Clause 4)

The microchannel device described in Clause 3, wherein a single gas permeable membrane covers the second openings included in the first group and the second openings included in the second group.

According to the microchannel device described in Clause 4, the number of gas permeable membranes that cover the second openings can be set to one.

(Clause 5)

The microchannel device described in Clause 1, wherein when the microchannel device is viewed in the plan view, the second openings included in the first group and the second openings included in the second group are arranged on respective sides of opposing sides of the microchannel device, and a single main channel is arranged in a central portion of the microchannel device.

According to the microchannel device described in Clause 5, a length of one main channel can be shorter.

(Clause 6)

The microchannel device described in Clause 1, wherein when the microchannel device is viewed in the plan view, the second openings included in the first group are arranged in a central portion of the microchannel device and the second openings included in the second group are arranged on a side of one side of the microchannel device, and a single main channel is arranged at a position that passes on an outer side of the first group and passes through the central portion of the microchannel device.

According to the microchannel device described in Clause 6, a length of one main channel can be shorter.

(Clause 7)

The microchannel device described in Clause 2, wherein the collection portion is a buffer space larger in volume than the main channel.

According to the microchannel device described in Clause 7, the collection portion is a buffer space larger in volume than the main channel, and hence the test solution that remains in the main channel can all be collected.

(Clause 8)

The microchannel device described in Clause 7, wherein the buffer space is provided with a water absorbing member.

According to the microchannel device described in Clause 8, a backflow from the collection portion to the main channel can be prevented and volatilization of the test solution from the main channel can be prevented.

(Clause 9)

The microchannel device described in any one of Clauses 1 to 8, wherein the microchannels are higher in channel resistance than the main channel.

According to the microchannel device described in Clause 9, the microchannel is higher in channel resistance than the main channel, and hence the test solution in the main channel can flow into the microchannels substantially at the same time.

(Clause 10)

The microchannel device described in any one of Clauses 2, 7, and 8, wherein the gas permeable membrane further covers the third opening.

According to the microchannel device described in Clause 10, possibility of discharge of the test solution that remains in the main channel through the third opening without remaining in the collection portion in discharge of the test solution to the collection portion can be lowered.

(Clause 11)

The microchannel device described in any one of Clauses 1 to 10, wherein the plurality of microchannels further include a third group.

According to the microchannel device described in Clause 11, a larger number of microchannels can be provided.

Though an embodiment of the present invention has been described, it should be understood that the embodiment disclosed herein is illustrative and non-restrictive in every respect. The scope of the present invention is defined by the terms of the claims and is intended to include any modifications within the scope and meaning equivalent to the terms of the claims.

What is claimed is:

1. A microchannel device used for a test in which a test solution containing a sample and an agent are to act on each other, the microchannel device having a plate shape, the microchannel device comprising:
a first opening for receiving the test solution that is to be injected therethrough;
a main channel through which the injected test solution can flow, the main channel including an inlet-side end that communicates with the first opening and an outlet-side end located opposite to the inlet-side end;
a plurality of microchannels each including a first-side end that communicates with the main channel and a second-side end located opposite to the first-side end;
second openings that each communicate with the second-side end of each of the microchannels;
a reservoir where the agent is to be stored, the reservoir being provided in each of the microchannels; and
a gas permeable membrane that covers at least one of the second openings, wherein
the plurality of microchannels include a first group and a second group,
the microchannels included in each of the first group and the second group are arranged as being aligned in a first direction when the microchannel device is viewed in a plan view, and
the first group and the second group are arranged as being aligned in a second direction orthogonal to the first direction.

2. The microchannel device according to claim 1, further comprising:
a collection portion where some of the test solution is collected, the collection portion being provided at the outlet-side end; and
a third opening provided in the collection portion.

3. The microchannel device according to claim 1, wherein
when the microchannel device is viewed in the plan view, the second openings included in the first group and the second openings included in the second group are arranged to face each other, and
a single main channel is arranged at a position surrounding an outer side of the plurality of microchannels.

4. The microchannel device according to claim 3, wherein
a single gas permeable membrane covers the second openings included in the first group and the second openings included in the second group.

5. The microchannel device according to claim 1, wherein
when the microchannel device is viewed in the plan view, the second openings included in the first group and the second openings included in the second group are arranged on respective sides of opposing sides of the microchannel device, and
a single main channel is arranged in a central portion of the microchannel device.

6. The microchannel device according to claim 1, wherein
when the microchannel device is viewed in the plan view, the second openings included in the first group are arranged in a central portion of the microchannel device and the second openings included in the second group are arranged on a side of one side of the microchannel device, and
a single main channel is arranged at a position that passes on an outer side of the first group and passes through the central portion of the microchannel device.

7. The microchannel device according to claim 2, wherein
the collection portion is a buffer space larger in volume than the main channel.

8. The microchannel device according to claim 7, wherein
the buffer space is provided with a water absorbing member.

9. The microchannel device according to claim 8, wherein
the microchannels are higher in channel resistance than the main channel.

10. The microchannel device according to claim 2, wherein
the gas permeable membrane further covers the third opening.

11. The microchannel device according to claim 1, wherein
the plurality of microchannels further include a third group.
